(19) United States
(12) Patent Application Publication
(10) Pub. No.: US 2019/0127700 A1
(43) Pub. Date: May 2, 2019

(54) Technique for Enhancing Stem Cell Regeneration Through Interaction of Vascular Endothelial Cell and Mesenchymal Stem Cell

(71) Applicant: The Catholic University of Korea Industry-Academic Cooperation Foundation, Seoul (KR)

(72) Inventors: Il Hoan OH, Seoul (KR); Jin A KIM, Seoul (KR)

(21) Appl. No.: 16/091,146

(22) PCT Filed: Apr. 5, 2017
(86) PCT No.: PCT/KR2017/003739

§ 371 Date: Oct. 4, 2018

(30) Foreign Application Priority Data
Apr. 6, 2016 (KR) 10-2016-0042201

(51) Int. Cl. C12N 5/0775; C12N 5/071

(52) U.S. Cl. CPC C12N 5/0662; C12N 5/069; C12N 2502/1352; C12N 2502/28; C12N 2501/415; C12N 2501/115; C12N 2501/15; C12N 2501/165; C12N 2501/2306; C12N 2501/231; C12N 2501/25; C12N 2501/24; C12N 2501/145

(57) ABSTRACT

In the present invention, it was discovered that when vascular endothelial cells are co-cultured with mesenchymal stem cells, an improvement is brought about in the niche activity of mesenchymal stem cells and the self-renewal of hematopoietic or neural stem cells and particularly that the niche activity and the self-renewal of hematopoietic or neural stem cells can easily be controlled by regulating the stimulus (cytokines, etc.) of vascular endothelial cells to the mesenchymal stem cells. Therefore, the present invention can not only improve the self-renewal of stem cells, but also easily control it as needed, and thus is expected to expand the usefulness of mesenchymal stem cell-based cell therapy.

### BACKGROUND

### 1. Field of the Invention

The present invention relates to a technique for enhancing stem cell regeneration by an interaction between vascular endothelial cells and mesenchymal stem cells.

### 2. Discussion of Related Art

In the 21^{st} century, biotechnology aims at the final goal of human welfare, and thus suggests the possibility of new solutions for food, environmental and health issues, and recently, the technology of stem cells is emerging as new technology for treating intractable diseases. Previously, organ transplantation and gene therapy have been suggested for the treatment of human intractable diseases, and efficient commercialization was difficult due to immune rejection, the shortage of donor organs, and the lack of knowledge about vector development or disease genes.

For this reason, as interest in pluripotent stem cells having an ability to form all organs through proliferation and differentiation has increased, stem cells are considered to be able to basically address organ damage as well as to treat most diseases, and many scientists have suggested the possibility of various applications of stem cells for treatment of an intractable disease such as Parkinson's disease, a variety of cancer, diabetes, and a spinal injury as well as regeneration of almost all organs.

Stem cells are cells capable of differentiating into various cells constituting biological tissue, and include undifferentiated cells in a stage earlier than differentiation, which can be obtained from each tissue of embryos, fetuses and adults. Among diverse categories of stem cells, pluripotent stem cells refer to stem cells with multi-functionality, which are able to differentiate into all of three types of germ layers constituting an organism. For categorization, stem cells may be categorized according to an anatomically-existing site, a cell function, the type of an antigen expressed on a cell surface, a transcription factor, a protein produced by a cell, or the specific type of cells that can be produced by the stem cells, and the most commonly used and relatively distinct categorization among diverse categorizations is performed according to a subject from which stem cells are isolated.

When isolated from an embryo, stem cells may be categorized as embryonic stem cells (ES cells), and when isolated from an adult, stem cells may be categorized as adult stem cells. According to another common classification, cells may be divided into pluripotent, multipotent and unipotent stem cells according to how many types of differentiated cells are produced from one stem cell, and generally, ES cells may be divided into pluripotent stem cells, and adult stem cells may be divided into multipotent and unipotent stem cells. The inner cell mass of a blastocyst formed in the early development after fertilization is the part that will form a fetus in the future, and ES cells formed from the inner cell mass may be pluripotent stem cells with a potential for differentiation into all tissues constituting one subject. In other words, ES cells are undifferentiated cells which can be unlimitedly proliferated, can differentiate into all cells and also differentiate into germ cells, unlike adult stem cells, thereby being genetically transferred to the next generation.

Multipotent stem cells were first isolated from adult bone marrow, and then also identified in various adult tissues. In other words, the bone marrow is the most widely known origin of stem cells, but multipotent stem cells may be acquired from skin, a blood vessel, a muscle, and a brain. However, stem cells are rarely present in adult tissue such as bone marrow. These cells are difficult to be cultured without induction of differentiation, and the culture of these cells is difficult without specifically-screened media. In other words, it is very difficult to isolate and ex vivo conserve the stem cells. For this reason, recently, there is great interest in identification, proliferation and differentiation of these human stem cells.

In addition, as the application of stem cells in tissue engineering, gene therapy and cell therapeutics has rapidly progressed, currently, interest in stem cells is explosively increasing in the art, and therefore, there is an eager demand for development of stem cells acquired from various tissues, technology associated with a method of proliferating stem cells which can exhibit a more stable and excellent proliferation effect, and a method for stably differentiating into various cells or tissues (Korean Unexamined Patent Application Publication No. 10-2012-00111301).

### SUMMARY OF THE INVENTION

The present invention is provided to solve the above-mentioned problems, the inventors had attempted to improve the effectiveness of mesenchymal stem cell-based cell therapy, and therefore discovered that when vascular endothelial cells are co-cultured with mesenchymal stem cells, an improvement is brought about in the niche activity of mesenchymal stem cells and the self-renewal of hematopoietic or neural stem cells and particularly that the niche activity can easily be controlled by regulating the stimulus (cytokines, etc.) of vascular endothelial cells with respect to the mesenchymal stem cells, and based on this, the present invention was completed.

An object of the present invention is directed to providing a composition for promoting the self-renewal of adult stem cells using vascular endothelial cells.

Another object of the present invention is directed to providing a method for preparing mesenchymal stem cells with improved niche activity, which includes co-culturing vascular endothelial cells and mesenchymal stem cells, which are isolated ex vivo, while being physically in contact with each other.

Still another object of the present invention is directed to providing a method of regulating the niche activity of mesenchymal stem cells using vascular endothelial cells.

However, technical problems to be solved in the present invention are not limited to the above-described problems, and other problems which are not described herein will be fully understood by those of ordinary skill in the art from the following descriptions.

To obtain the objects of the present invention, the present invention provides a composition for promoting the self-renewal of adult stem cells, which includes vascular endothelial cells over-expressing any one or more of ligands Wnt and EphrinB2.

In one exemplary embodiment of the present invention, the vascular endothelial cells may have increased STAT3 activity.

In another exemplary embodiment of the present invention, the vascular endothelial cells may be transfected with a vector including any one of a Wnt ligand, an EphrinB2 ligand, activated STAT3 (STAT3-C) and a combination thereof, and treated with any one factor selected from a basic fibroblast growth factor (b-FGF), thrombopoietin (TPO), a vascular endothelial growth factor (VEGF), interleukin-6 (IL-6), interleukin-10 (IL-10), tumor necrosis factor-α (TNF-α) and a combination thereof.

In still another exemplary embodiment of the present invention, the composition may further include ex-vivo isolated mesenchymal stem cells, and the mesenchymal stem cells may be provided while being in physical contact with vascular endothelial cells.

In addition, the present invention provides a composition for promoting the self-renewal of adult stem cells, which includes vascular endothelial cells; and any one factor selected from the group consisting of b-FGF, TPO, VEGF, IL-6, IL-10, TNF-α, and a combination thereof.

In one exemplary embodiment of the present invention, the composition may further include ex-vivo isolated mesenchymal stem cells, and the mesenchymal stem cells may be provided while being in physical contact with vascular endothelial cells.

In addition, the present invention provides a method of preparing mesenchymal stem cells with improved niche activity, which includes co-culturing vascular endothelial cells and mesenchymal stem cells, which are isolated ex vivo, while being in physical contact with each other.

In one exemplary embodiment of the present invention, before co-culture, over-expressing any one of a Wnt ligand, an EphrinB2 ligand, activated STAT3 (STAT3-C) and a combination thereof in the vascular endothelial cells may be further included.

In another exemplary embodiment of the present invention, the co-culturing may include adding any one factor selected from the group consisting of b-FGF, TPO, VEGF, IL-6, IL-10, TNF-α and a combination thereof.

In addition, the present invention provides a method of regulating the niche activity of mesenchymal stem cells, which includes over-expressing or under-expressing any one or more of ligands Wnt and EphrinB2 in vascular endothelial cells, which are co-cultured while being in physical contact with ex-vivo isolated mesenchymal stem cells.

In one exemplary embodiment of the present invention, the over-expressing may include transfecting vascular endothelial cells with a vector including any one of a Wnt ligand, an EphrinB2 ligand, an activated STAT3 (STAT3-C) and a combination thereof, or by treating vascular endothelial cells with a factor selected from the group consisting of b-FGF, TPO, VEGF, IL-6, IL-10, TNF-α and a combination thereof.

In another exemplary embodiment of the present invention, the under-expressing may include treating vascular endothelial cells with a factor selected from the group consisting of a transforming growth factor, β3 (Tgfβ3), interferon gamma (IFN-γ) and a combination thereof.

Meanwhile, in the present invention, the mesenchymal stem cells may be derived from adipose tissue, bone marrow, peripheral blood or umbilical cord blood, and the adult stem cells may be hematopoietic stem cells or neural stem cells.

In the present invention, it was discovered that when vascular endothelial cells are co-cultured with mesenchymal stem cells, an improvement is brought about in the niche activity of mesenchymal stem cells and the self-renewal of hematopoietic or neural stem cells and particularly that the niche activity and the self-renewal of hematopoietic or neural stem cells can easily be controlled by regulating the stimulus (cytokines, etc.) of vascular endothelial cells to the mesenchymal stem cells. Therefore, the present invention can not only improve the self-renewal of stem cells, but also easily regulate it as needed, and thus is expected to expand the usefulness of mesenchymal stem cell-based cell therapy.

### BRIEF DESCRIPTION OF THE DRAWINGS

FIG. 1 is a schematic diagram illustrating an experimental process for showing the changes in characteristics of mesenchymal stem cells due to co-culture with vascular endothelial cells.

FIG. 2 shows the result of comparing the (a) morphological characteristic, (b) size, and (C) cell proliferation of mesenchymal stem cells to confirm a change in cell growth of mesenchymal stem cells co-cultured with vascular endothelial cells.

FIG. 3 shows the result of comparing the (a) cell cycle; and (b) cell cycle regulators of mesenchymal stem cells to confirm a change in cell growth of mesenchymal stem cells co-cultured with vascular endothelial cells.

FIG. 4 shows the result of comparing (a) CFU-Fs; and (b) differentiation into adipose tissue in mesenchymal stem cells to confirm the enhancement in stemness of mesenchymal stem cells co-cultured with vascular endothelial cells.

FIG. 5 shows the results of comparing the (A) size; (B) cell proliferation; (C) CFU-Fs; and (D) osteogenesis or differentiation into adipose tissue in mesenchymal stem cells between a group cultured without contact with vascular endothelial cells (EC-CM) and a group in which mesenchymal stem cells were cultured alone (MSC-CM).

FIG. 6 shows the result of comparing the expression of genes associated with perivascular characteristics to confirm a change in cellular characteristics of mesenchymal stem cells co-cultured with vascular endothelial cells.

FIG. 7 shows the result of comparing (A) EMT genes; (B) EMC inducer genes; (C) EMT inducer-receptor genes; and pluripotency-associated genes in mesenchymal stem cells to confirm changes in cellular characteristics of mesenchymal stem cells co-cultured with vascular endothelial cells.

FIG. 8 shows (a) the schematic diagram of an experimental process; (b) hematopoietic supporting activity (LSK cells); and (c) the expression of self-renewal markers to confirm an effect of enhancing the niche activity of mesenchymal stem cells co-cultured with vascular endothelial cells.

FIG. 9 shows (A) the expression of hematopoietic cytokines; and (B) the expression of notch ligands in mesenchymal stem cells to confirm an effect of enhancing the niche activity of mesenchymal stem cells co-cultured with vascular endothelial cells.

FIG. 10 shows gene expression of (a) notch reporters, and (b) notch targets in co-cultured hematopoietic stem cells to confirm an effect of enhancing the niche activity of mesenchymal stem cells co-cultured with vascular endothelial cells.

FIG. 11 shows the expression of (A) b-catenin; (B) b-catenin co-factors; (C) Wnt receptors; and (C) b-catenin regulators in co-cultured vascular endothelial cells to identify expression factors of niche activity-associated vascular endothelial cells.

FIG. 12 shows (A) Wnt protein expression in co-cultured vascular endothelial cells; (B) EphrinB2 and ErB4 expression in co-cultured mesenchymal stem cells; and (C) a schematic diagram illustrating the mechanism associated with the self-renewal of hematopoietic stem cells according to the present invention to identify expression factors of niche activity-associated vascular endothelial cells.

FIG. 13 shows (a) the result of comparing Wnt ligand expression in co-cultured vascular endothelial cells; and (b) the result of comparing notch ligand expression in co-cultured mesenchymal stem cells according to cytokine treatment to identify a signaling factor controlling a stimulatory effect of vascular endothelial cells.

FIG. 14 shows (A) a schematic diagram of a retrovirus vector expressing activated STAT3 (STAT3-C; SC) and inhibited STAT3 (dnSTAT3); (B) the result of indicating the expression of notch ligands of activated STAT3-introduced vascular endothelial cells; (C) the result of indicating CFU-Fs of vascular endothelial cell-cocultured mesenchymal stem cells; and (D) the result of indicating an effect of the mesenchymal stem cells for improving self-renewal of hematopoietic stem cells to identify a signaling factor controlling a stimulatory effect of vascular endothelial cells.

FIG. 15 is a schematic diagram illustrating a mechanism of controlling self-renewing capacity in hematopoietic stem cells according to the present invention.

FIG. 16 shows the result of analyzing the function of a motor nerve by a Basso Mouse Scale (BMS) method, indicating an effect of promoting nervous system self-renewal by co-administration of mesenchymal stem cells and vascular endothelial cells into spinal cord injury animal models.

FIG. 17 shows the result of analyzing the function of a sensory nerve by a somatosensory evoked potential (SEP) method, indicating an effect of promoting nervous system self-renewal by co-administration of mesenchymal stem cells and vascular endothelial cells into spinal cord injury animal models.

FIG. 18 shows the result of comparing NeuN-positive cell numbers per spinal cord-damaged site to confirm a self-renewal effect on the nervous system by co-administration of mesenchymal stem cells and vascular endothelial cells into spinal cord injury animal models.

FIG. 19 shows the result of comparing Tuj-1-positive cell numbers per spinal cord damage site to confirm a self-renewal effect for the nervous system by co-administration of mesenchymal stem cells and vascular endothelial cells into spinal cord injury animal models.

FIG. 20 is a result of comparing nestin-positive cell numbers per spinal cord damage site to confirm a self-renewal effect for the nervous system by co-administration of mesenchymal stem cells and vascular endothelial cells into spinal cord injury animal models.

FIG. 21 is a schematic diagram illustrating a method of regulating the self-renewal of hematopoietic or neural stem cells using the technology of the present invention.

### DETAILED DESCRIPTION OF EXEMPLARY EMBODIMENTS

Hereinafter, the present invention will be described in detail.

The present invention provides a composition for promoting the self-renewal of adult stem cells, which includes vascular endothelial cells over-expressing any one or more of ligands Wnt and EphrinB2; a method of promoting the self-renewal of adult stem cells, which includes administering vascular endothelial cells over-expressing any one or more of ligands Wnt and EphrinB2 to a subject; and a use of vascular endothelial cells over-expressing any one or more of ligands Wnt and EphrinB2 for promoting the self-renewal of adult stem cells.

The term “self-renewal” used herein is an ability to produce cells having the same characteristics and features, is also called self-replication, self-reproduction or self-regeneration, and one of the most significant features of stem cells. Particularly, the self-renewal in the present invention refers to an ability to proliferate cells maintained in an undifferentiated state.

The term “adult stem cells” used herein may be multipotent and unipotent stem cells, also acquired from the skin, blood vessels, muscles and brain. However, stem cells in adult tissue such as the bone marrow are very rarely present, and are difficult to be cultured without induction of differentiation. These cells are difficult to be cultured without specifically-screened media. In other words, stem cells are very difficult to be isolated and ex vivo conserved. Meanwhile, for the purpose of the present invention, the adult stem cells may be hematopoietic stem cells or neural stem cells.

The term “hematopoietic stem cells (HSCs)” used herein refers to ancestor cells of undifferentiated bone marrow hematopoietic cells generating blood cells such as erythrocytes, leukocytes, and thrombocytes, and also called hematopoietic progenitor cells. The term “neural stem cells (NSCs)” refers to primitive cells present in the nervous system, and undifferentiated stem cells generating nerve cells such as astrocytes, neurons and oligodendrocytes. The hematopoietic stem cells or neural stem cells have self-renewing capacity when transplanted into a host having destroyed bone marrow or a destroyed nervous system, and exhibit long-term repopulation. The adult stem cells of the present invention include adult stem cells derived from all animals such as humans, monkeys, pigs, horses, cows, sheep, dogs, cats, mice, rats, etc., and preferably human-derived cells.

In the present invention, it was demonstrated that, as vascular endothelial cells and mesenchymal stem cells are co-cultured while being in physical contact with each other, the niche activity of mesenchymal stem cells may be enhanced, and thus the self-renewing capacity of adult stem cells may be enhanced. Particularly, it was experimentally confirmed that an effect caused by an interaction between vascular endothelial cells and mesenchymal stem cells results from the Wnt ligand and the EphrinB2 ligand of vascular endothelial cells. Therefore, the present invention has a technical characteristic in that technology of enhancing the niche activity of mesenchymal stem cells using vascular endothelial cells over-expressing any one or more of ligands Wnt and EphrinB2, specifically, technology of promoting the self-renewal of adult stem cells, is provided.

In the present invention, by using a method known in the art, a Wnt or EphrinB2 ligand may be over-expressed in vascular endothelial cells, and preferably it may be over-expressed by transfecting vascular endothelial cells with a vector containing any one of a Wnt ligand, an EphrinB2 ligand, activated STAT3 (STAT3-C), and a combination thereof, or treating vascular endothelial cells with any one factor selected from the group consisting of b-FGF, TPO, VEGF, IL-6, IL-10, TNF-α, and a combination thereof.

The term “vector” used herein refers to an expression vector into which a polynucleotide sequence encoding the factor is inserted, and preferably a plasmid, virus or another mediator, which is known in the art, but the present invention is not limited thereto. The polynucleotide sequence according to the present invention may be operably linked to an expression control sequence, and the gene sequence and the expression control sequence, which are operably linked, may be included in one expression vector including a selection marker and a replication origin as well. The “operably linked” means that, when a suitable molecule binds to an expression control sequence, a gene is linked to the expression control sequence in such a manner that expression can occur. The “expression control sequence” refers to a DNA sequence which controls the expression of a polynucleotide sequence operably linked to specific host cells. Such a control sequence includes a promoter for performing transcription, an arbitrary operator sequence for controlling transcription, a sequence encoding a suitable mRNA ribosome-binding site and a sequence controlling the termination of transcription and translation.

Examples of the plasmids may include E. coli-derived plasmids (pBR322, pBR325, pUC118 and pUC119), Bacillus subtilis-derived plasmids (pUB110 and pTP5) and yeast-derived plasmids (YEp13, YEp24 and YCp50), and the virus may be selected from a complex with a retrovirus, an adenovirus, a vaccinia virus, a baculovirus, a herpes simplex virus and a lentivirus. One of ordinary skill in the art may use a suitable vector to introduce a gene or polynucleotide of the present invention into host cells, and preferably, a vector designed to facilitate induction of protein expression and isolation of an expressed protein may be used.

Meanwhile, the present invention may be modified and applied within a range capable of accomplishing the same function as described above. First, as described above, by targeting in-vivo-existing mesenchymal stem cells, as described above, only vascular endothelial cells over-expressing any one or more of ligands Wnt and EphrinB2 may be provided. In addition, combinations of vascular endothelial cells and cytokines (b-FGF, TPO, VEGF, IL-6, IL-10, TNF-α, and a combination thereof) capable of enhancing the expression of a Wnt or EphrinB2 ligand may be provided. Moreover, ex-vivo isolated mesenchymal stem cells may be added to the two types of compositions, and at this time, the vascular endothelial cells and the mesenchymal stem cells may be transplanted while being in physical contact with each other, or, if necessary, may be injected in vivo after co-culture.

The term “mesenchymal stem cells (MSCs)” used herein are cells which serve as the origin for creating cartilage, bone, fat, bone marrow stroma, muscle, nerve, etc., and in adults, are generally present in the bone marrow, but also present in umbilical cord blood, peripheral blood, and other tissues, and thus are obtained therefrom. In the specification, MSCs are used in the same sense as mesenchymal stromal or stromal cells. The MSCs include cells derived from all animals such as humans, monkeys, pigs, horses, cows, sheep, dogs, cats, mice, and rats, and preferably human-derived cells.

The term “niche” used herein refers to a component (cells and/or a material) consisting of tissues or organs supporting development and proliferation of tissue cells such as stem cells and somatic cells, other than the stem cells, and the niche has been known to secrete factors required for inducing cellular interactions and having totipotency. The niche is also called a microenvironment, and plays a critical role in retaining stemness expressing all characteristics of stem cells. Stem cells are anchored in a type of microenvironment consisting of adhesion molecule growth factors, which is called niche in academic circles. Such a region of a stem cell serves to support and regulate a location, adhesiveness, homing, quiescence, and activation. In other words, the niche is considered as a major microenvironment that surrounds stem cells serving to regulate differentiation of stem cells, and prevent and protect migration to another site or apoptosis.

The composition for promoting the self-renewal of adult stem cells according to the present invention may be used to treat a patient requiring transplantation of hematopoietic stem cells. Specifically, the composition may be used for transplantation into a patient with acute leukemia, chronic myelogenous leukemia, myelodysplastic syndrome, lymphoma, multiple myeloma, a germ cell tumor, breast cancer, ovarian cancer, small cell lung cancer, neuroblastoma, aplastic anemia, erythropathy, Gaucher's disease, Hunter syndrome, adenosine deaminase (ADA) deficiency, Wiskott-Aldrich syndrome, rheumatoid arthritis, systemic lupus erythematosus, or multiple sclerosis, or a patient with damaged hematopoietic cells due to chemotherapy or radiation therapy.

In addition, the composition for promoting the self-renewal of adult stem cells according to the present invention may be used to treat a patient requiring the transcription of neural stem cells, and specifically, may be used for transplantation into a patient with a degenerative nervous system disease selected from the group consisting of a stroke, Alzheimer's disease, Parkinson's disease, Huntington's disease, Amyotrophic lateral sclerosis (ALS), Pick's disease, Niemann-Pick disease and a spinal cord injury/disease.

In the present invention, it was identified that the above-described effect of the interaction between the vascular endothelial cells and the mesenchymal stem cells results from signaling systems of the vascular endothelial cells itself, and the signal control with respect to the vascular endothelial cells may be applied as a method of amplifying the niche activity of the mesenchymal stem cells. Therefore, the present invention has another technical characteristic in that the niche activity of the mesenchymal stem cells may be enhanced by the increase in factors (Wnt and EphrinB2) of the vascular endothelial cells.

In another aspect of the present invention, the present invention provides a method of preparing mesenchymal stem cells with improved niche activity, which includes co-culturing vascular endothelial cells and mesenchymal stem cells, which are isolated ex vivo, while being in physical contact with each other.

Since the method of preparing mesenchymal stem cells with improved niche activity uses vascular endothelial cells and mesenchymal stem cells, which have been described in the description of the composition for promoting the self-renewal of adult stem cells, common descriptions will be omitted to avoid excessive complexity in the specification.

The present invention may further include, before co-culture, over-expressing any one of a Wnt ligand, an EphrinB2 ligand, activated STAT3 (STAT3-C), and a combination thereof in vascular endothelial cells, and the co-culturing may be performed by adding any one selected from the group consisting of b-FGF, TPO, VEGF, IL-6, IL-10, TNF-α and a combination thereof.

The over-expressing may be introduction of a vector containing any one gene selected from the group consisting of a Wnt ligand, an EphrinB2 ligand and activated STAT3 (STAT3-C) into vascular endothelial cells, but the present invention is not limited thereto.

The vector according to the present invention may be introduced into cells using a method known in the art, and a method of introducing a recombinant vector into host cells according to the present invention may include, but is not limited to, a calcium chloride (CaCl₂) and heat shock method, silicon carbide whiskers, sonication, precipitation by polyethylene glycol (PEG), transient transfection, microinjection, transduction, cell fusion, calcium phosphate precipitation, liposome-mediated transfection, DEAE-dextran-mediated transfection, polybrene-mediated transfection, electroporation or a gene gun method. The vector may be introduced into cells by another known method for injecting a nucleic acid into cells.

In the present invention, it was identified that the effect of the interaction between the vascular endothelial cells and the mesenchymal stem cells is caused by control of a signal system of vascular endothelial cells, and the control of a signal with respect to the vascular endothelial cells can be applied as a method of regulating the niche activity of the mesenchymal stem cells. Therefore, the present invention has a still another technical characteristic in that the niche activity of the mesenchymal stem cells can be up- or down-regulated by the control of factors (Wnt and EphrinB2) of the vascular endothelial cells.

Therefore, the present invention provides a method of regulating the niche activity of mesenchymal stem cells, which includes: over- or under-expressing any one or more of ligands Wnt and EphrinB2 in vascular endothelial cells, which are co-cultured with mesenchymal stem cells while being in physical contact therewith.

Since the method of regulating the niche activity of the mesenchymal stem cells according to the present invention uses vascular endothelial cells and mesenchymal stem cells, which have been described in the above-described composition for promoting the self-renewal of adult stem cells, common descriptions will be omitted to avoid excessive complexity in the specification.

In the present invention, the over-expressing may be performed by transfecting the vascular endothelial cells with a vector including any one of a Wnt ligand, an EphrinB2 ligand, activated STAT3 (STAT3-C) and a combination thereof, or treating the vascular endothelial cells with a factor selected from the group consisting of b-FGF, TPO, VEGF, IL-6, IL-10, TNF-α and a combination thereof.

In the present invention, the under-expressing may be performed by treating the vascular endothelial cells with a factor selected from the group consisting of Tgfb3, IFN-γ, and a combination thereof.

Hereinafter, exemplary examples will be presented to help in understanding of the present invention. However, the following examples are merely provided to more easily understand the present invention, and the scope of the present invention is not limited by the following examples.

### Example 1. Changes in Characteristics of Mesenchymal Stem Cells According to Co-Culture with Vascular Endothelial Cells

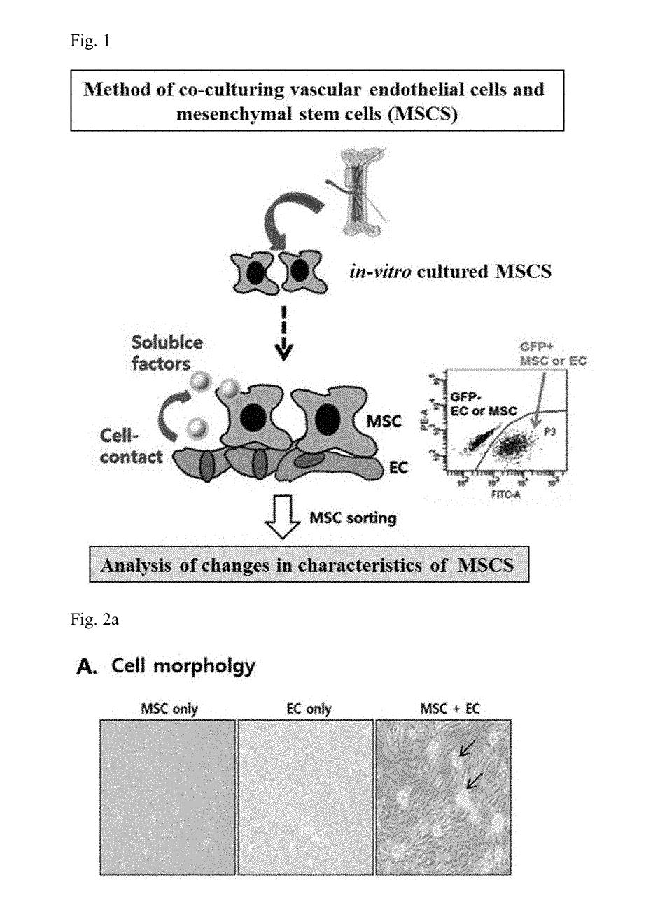

To confirm an effect of the vascular endothelial cells on the stemness of the mesenchymal stem cells, two types of cells were co-cultured in vitro, and the changes according thereto were analyzed. To isolate the co-cultured mesenchymal stem cells using a cell sorter, GFP was introduced only into one of the two types of cells. To design conditions similar to an in-vivo three-dimensional interaction between two cells, a method of plating the vascular endothelial cells on the bottom, and then plating the mesenchymal stem cells thereon was used. The conditions were designed in such a manner that direct cell-contact with the vascular endothelial cells and the effects caused by soluble factors affect mesenchymal stem cells by the co-culturing method (FIG. 1).

1-1. Change in Growth of Mesenchymal Stem Cells

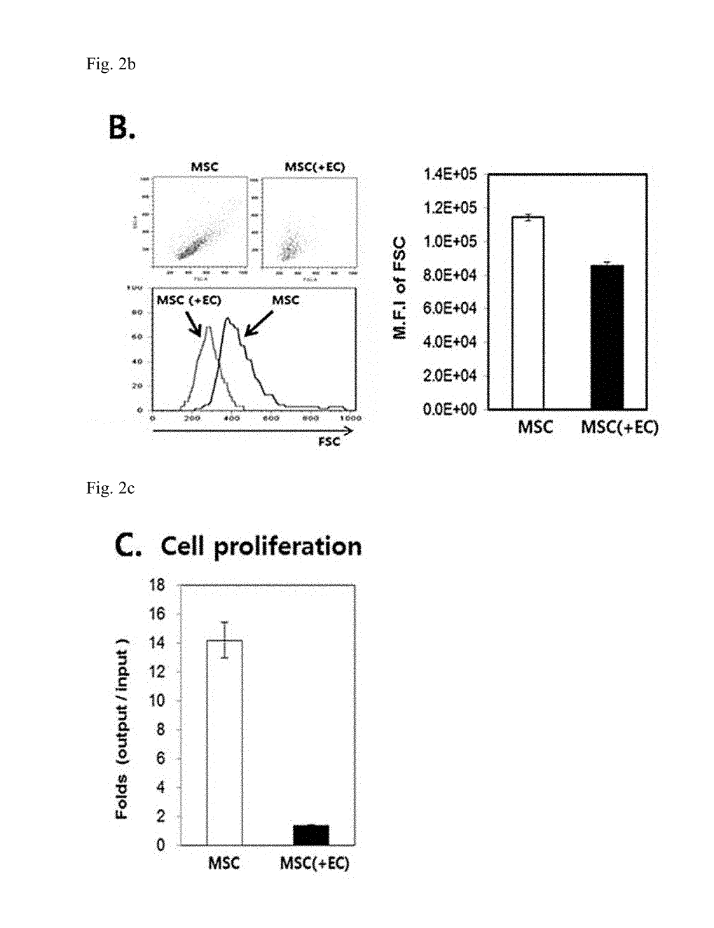

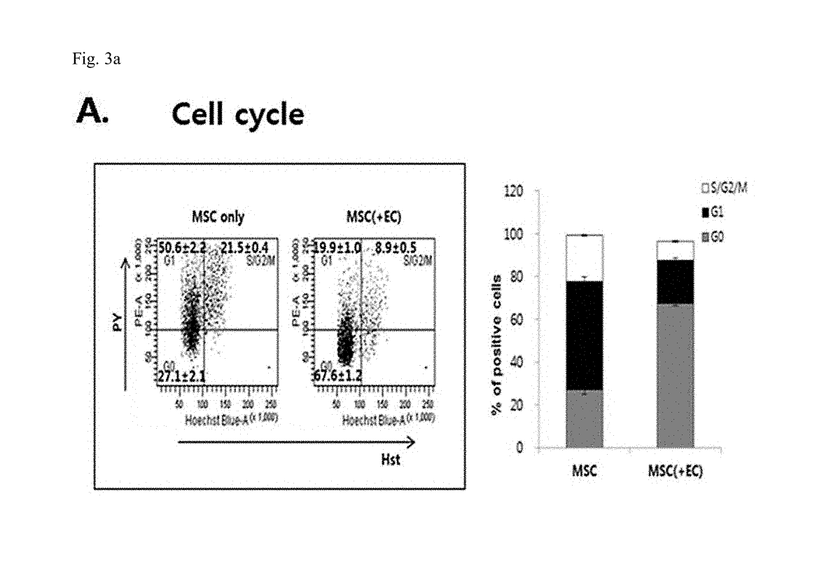

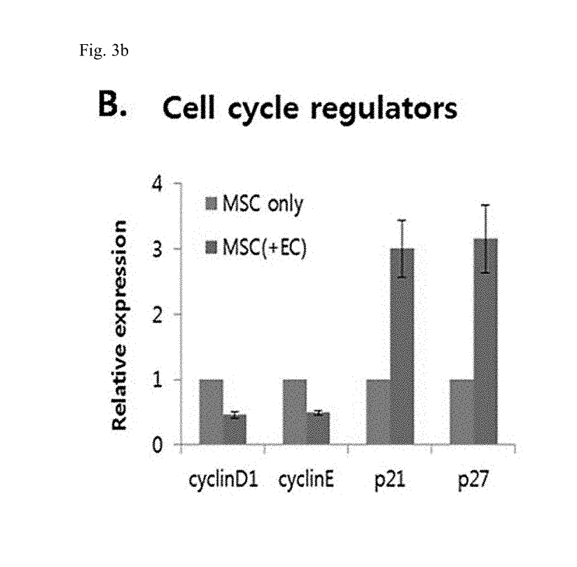

In this embodiment, a change in growth of mesenchymal stem cells by an interaction with vascular endothelial cells was to be confirmed, and as a comparative group, a group in which mesenchymal stem cells were cultured alone was used. It was confirmed that, unlike the single culture, mesenchymal stem cells co-cultured with vascular endothelial cells were grown on vascular endothelial cells in a sphere-like type, the size of cells was reduced, and cell proliferation was considerably reduced (FIGS. 2a to 2c). In addition, as a result of analyzing a cell cycle, compared with single culture, mesenchymal stem cells co-cultured with vascular endothelial cells exhibited high G0 arrest, thereby inhibiting overall cell cycle progression and maintaining quiescence (FIG. 3a). In addition, it was confirmed that the expression of G0-G1 progression-associated factors such as cyclinD1, E, etc. was reduced, and inhibitory factors such as p21, p27, etc. were increased (FIG. 3b).

1-2. Improvement in Stemness of Mesenchymal Stem Cells

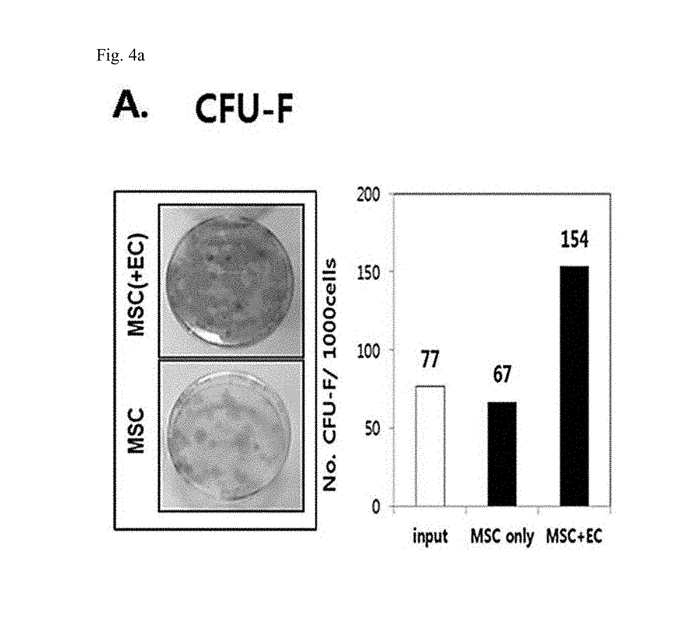

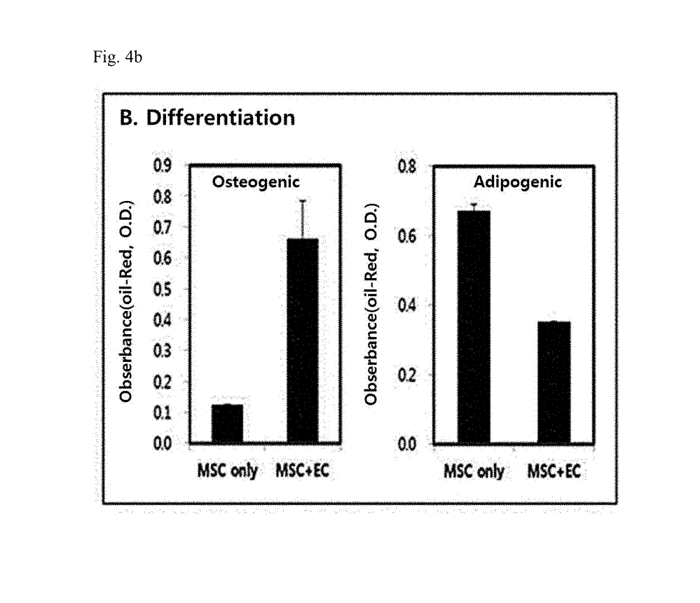

In this embodiment, a change in stemness of mesenchymal stem cells by an interaction with vascular endothelial cells was to be confirmed, and as a comparative group, and a group in which mesenchymal stem cells were cultured alone was used. The mesenchymal stem cells co-cultured with the vascular endothelial cells were increased in colony-forming unit fibroblasts (CFU-Fs), compared to the comparative group, and particularly, although cell proliferation is considerably reduced, as the result of increasing a ratio of the cells with CFU-Fs, compared to that before co-culture, it can be seen that an undifferentiated state was not only maintained, but also reprogramming to cells forming colonies occurs (FIG. 4a). In addition, in terms of the multipotency of the mesenchymal stem cells, differentiation into adipose cells tended to be inhibited by co-culture with vascular endothelial cells (FIG. 4b).

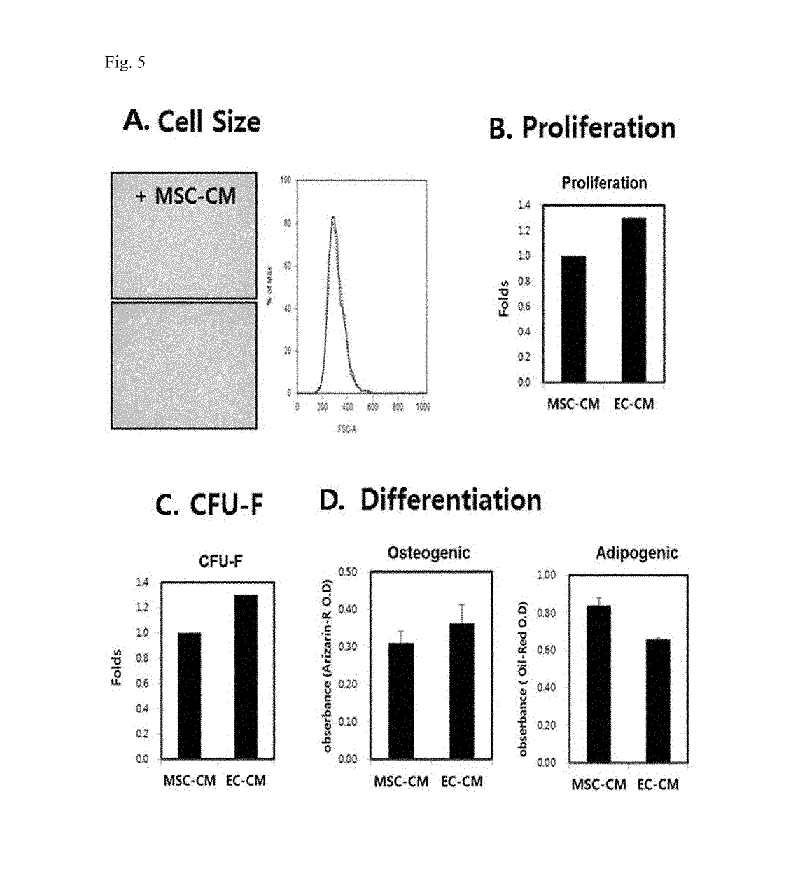

Moreover, to analyze an interaction mechanism with respect to the influence of vascular endothelial cells, the case of treatment only with a conditioned medium (EC-CM) for vascular endothelial cells (the group in which mesenchymal stem cells were cultured while not in contact with vascular endothelial cells) and the case in which mesenchymal stem cells were cultured alone (MSC-CM) were compared. As a result, since there were no differences in the size, growth, CFU-Fs, and differentiation capacity of mesenchymal stem cells between these groups, it was seen that the stemness is the phenomenon resulting from direct-contact between the two types of cells (FIGS. 5A to 5D).

1-3. Change in Cellular Characteristics of Mesenchymal Stem Cells

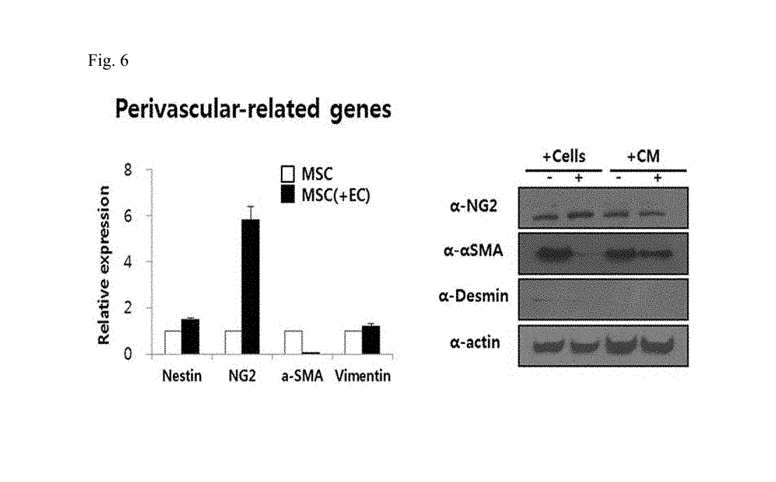

In this embodiment, a change in cellular characteristics of mesenchymal stem cells due to an interaction with vascular endothelial cells was to be confirmed, and a group in which mesenchymal stem cells were cultured alone was used. The mesenchymal stem cells co-cultured with vascular endothelial cells were increased in the expression of nestin and NG2 of markers for perivascular cells or pericytes, which are adjacent to vascular endothelial cells in vivo, but decreased in α-SMA, thereby exhibiting characteristics of arterial perivascular cells. This result shows that ex-vivo cultured mesenchymal stem cells acquire a perivascular character similar to that in vivo due to an interaction with vascular endothelial cells (FIG. 6).

In addition, in recent years, it can be confirmed that the co-cultured mesenchymal stem cells were considerably increased in expression of EMT markers (Sox9, Snaill, Zeb1, Twist, etc.) known to be associated with characteristics of undifferentiated mesenchymal stem cells as well as cancer stem cells, and reinforced in mesenchymal characteristics in terms of reprogramming of high-functional stemness (FIG. 7A). Indeed, in the vascular endothelial cells co-cultured with the mesenchymal stem cells, the expression of EMT-inducible cytokines was increased, and in the mesenchymal stem cells, the expression of IL-8 receptors (CXCR1, 2) was dramatically increased, indicating that the susceptibility of these factors was increased in the vascular endothelial cells. Therefore, it can be confirmed that, in the reprogramming of mesenchymal stromal cells resulting from an interaction between the two cells, EMT occurred (FIGS. 7B to 7C).

Meanwhile, in terms of the enhancement in stemness of mesenchymal stem cells, it can be seen that the expression of pluripotent genes such as Oct4, Sox2 and Klf4, which are involved in embryonic stemness or reprogramming of somatic cells, and an epigenetic regulator Chdl was increased, indicative of reprogramming of the cellular characteristics of the mesenchymal stem cells in a pluripotent state (FIG. 7D).

### Example 2. Confirmation of Effect of Promoting the Self-Renewal of Hematopoietic Stem Cells

2-1. Enhanced Niche Activity of Mesenchymal Stem Cells

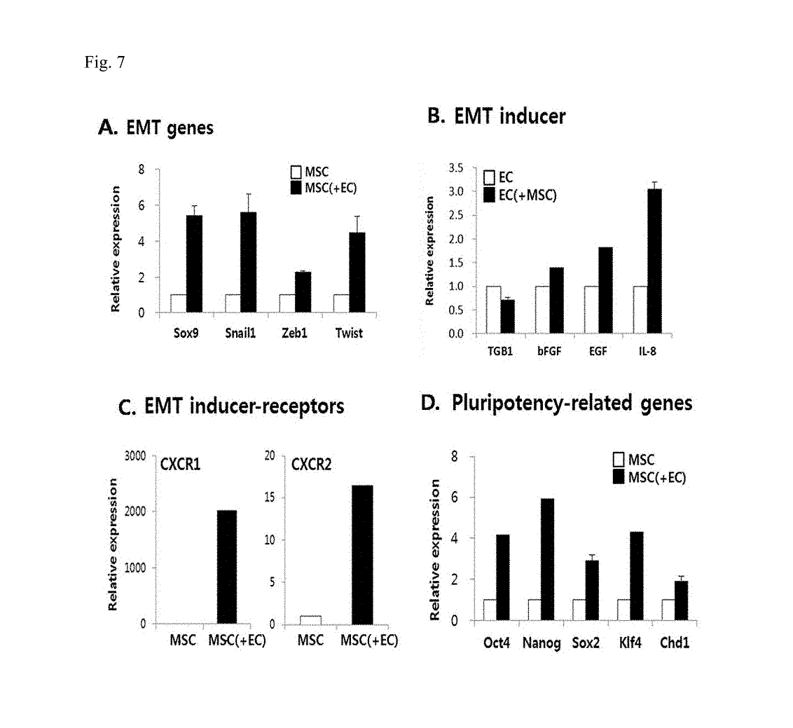

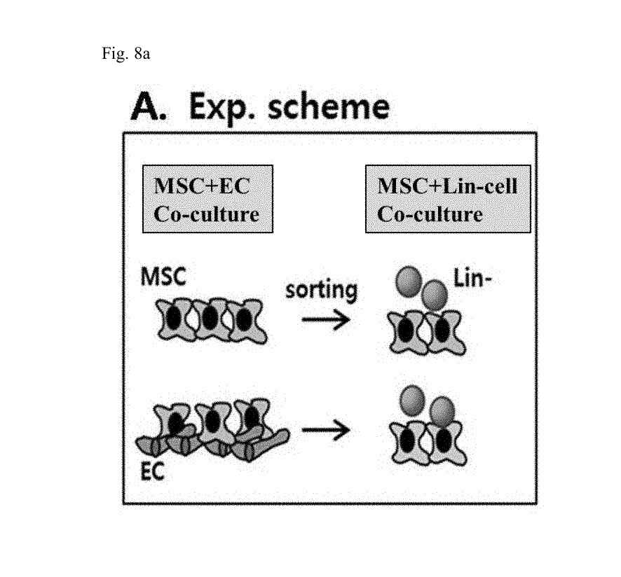

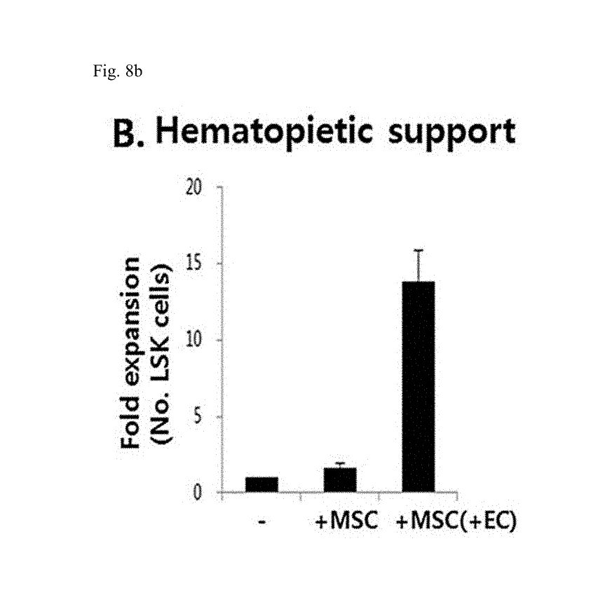

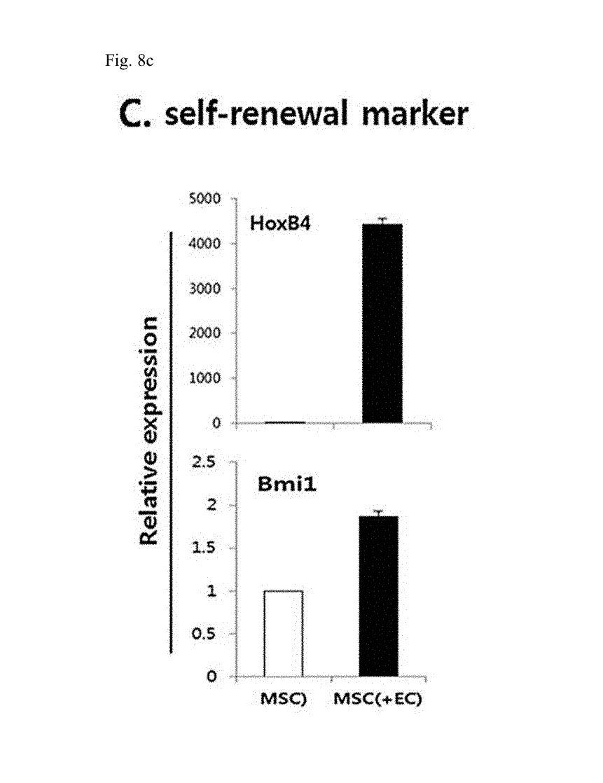

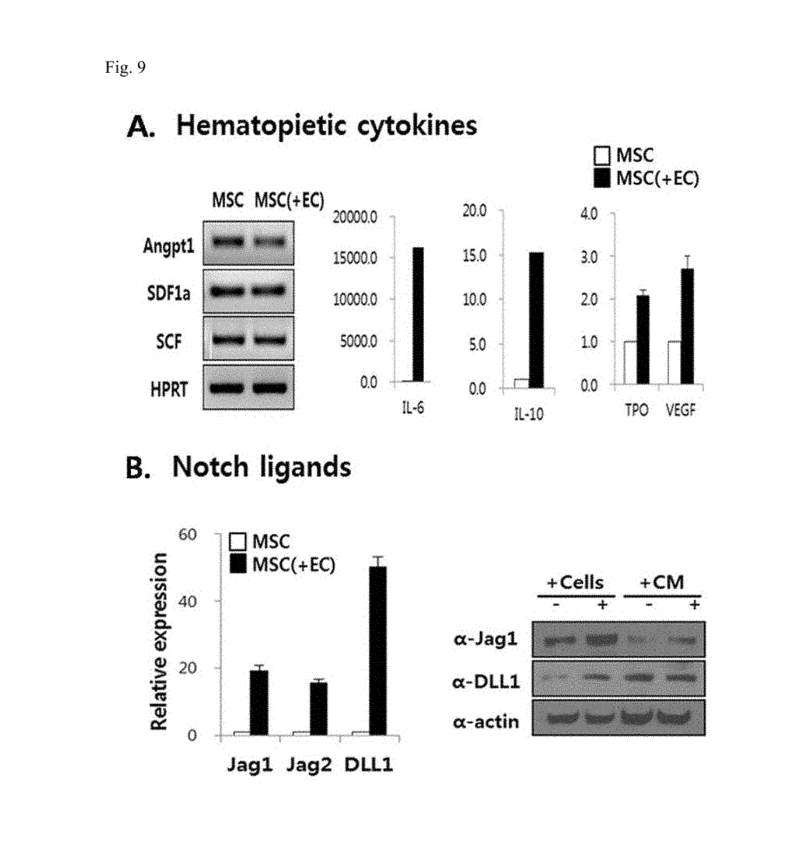

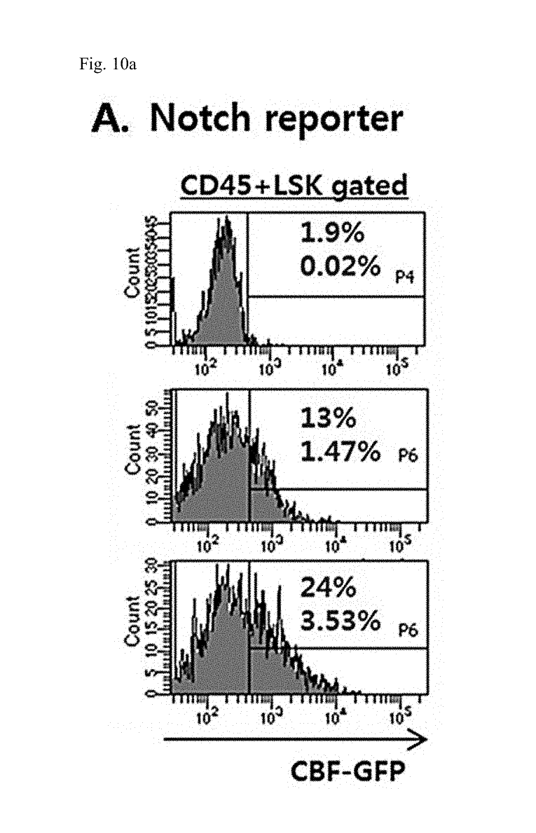

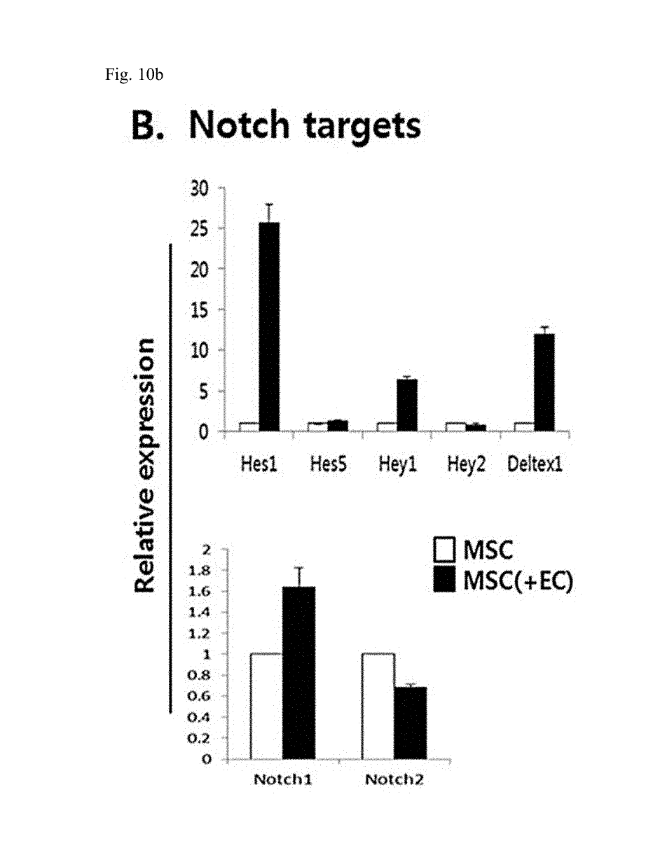

As a result of co-culture of isolated mesenchymal stem cells which were modified by the co-culture with the vascular endothelial cells and hematopoietic stem cells (Lin-cells) which were isolated from bone marrow, compared to single-cultured mesenchymal stem cells, the proliferation of undifferentiated hematopoietic stem cells Lin-Sca+Kit+(LSK cells) was promoted, and at this time, the expression of factors (HoxB4, Bmi1) involved in self-renewal of hematopoietic stem cells was also increased, indicating that niche activity was enhanced (FIGS. 8a to 8c). In addition, in the mesenchymal stem cells co-cultured with the vascular endothelial cells, the expression of cytokines (IL-6, IL-10, TPO and VEGF) known as growth factors of hematopoietic stem cells was increased, and particularly, the expression of notch ligands (Jagl, 2, and DLL1) known as major factors for the maintenance of undifferentiation and self-renewal of hematopoietic stem cells was increased (FIGS. 9A and 9B). Moreover, as a result of analysis using a CBF-reporter (a receptor for monitoring the function of CBF-1 (RBPJ-kappa) binding to NICD in notch signaling), it was confirmed that, in hematopoietic stem cells which were co-cultured with mesenchymal stem cells modified by vascular endothelial cells, notch signaling and the expression of notch target genes (Hes1, 5, Hey1 and Deltex1) were increased, reconfirming the enhanced niche activity of the mesenchymal stem cells (FIGS. 10a and 10b).

2-2 Confirmation of Expression Factors of Vascular Endothelial Cells Involved in Niche Activity

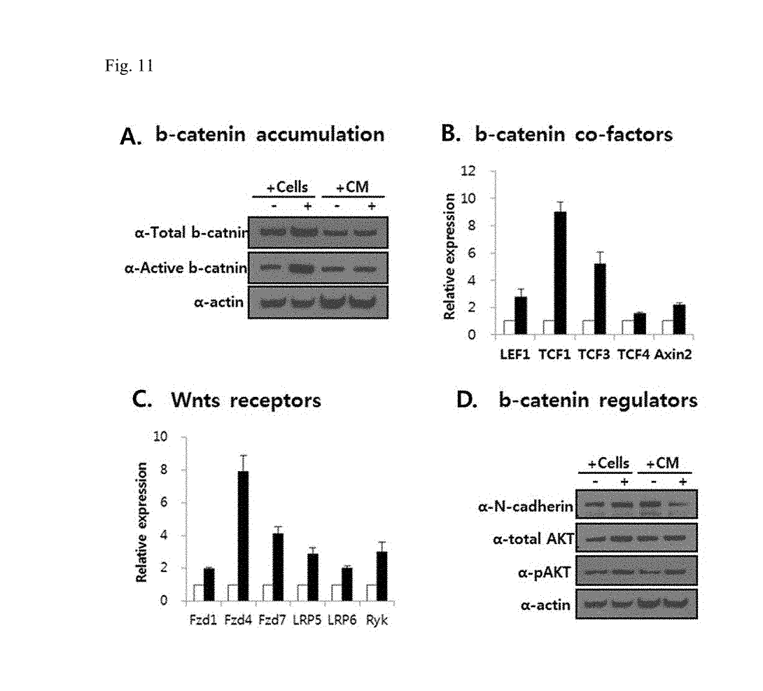

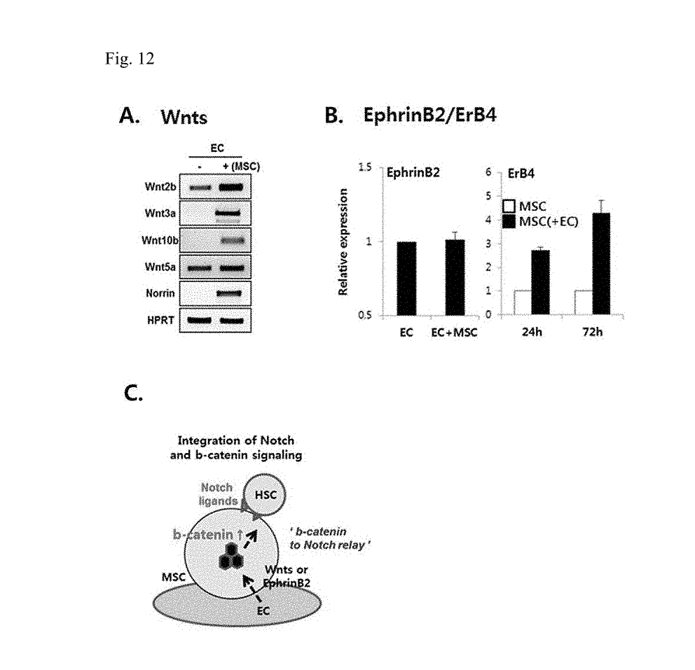

As a result of analyzing the mechanism of inducing the functional change in mesenchymal stem cells, specifically, the changes in major signaling factors, it was confirmed that co-factors LEF1, TCF1 and TCF3, which help b-catenin activity, were increased in mesenchymal stem cells (MSCs), and receptors (Fzd4, 7, and LRP family) capable of responding to a Wnt ligand were increased (FIGS. 11A to 11D). In addition, an EphrinB2 ligand known to be involved in stimulation of neural stem cells was expressed in vascular endothelial cells, and in this regard, the expression of a receptor ErB4, which is capable of responding to EphrinB2, was increased in mesenchymal stem cells. Therefore, it was found that, the Wnt ligand and EphrinB2 ligand of vascular endothelial cells are involved in a process of exhibiting the supporting activity of mesenchymal stem cells as mediators (FIGS. 12A to 12C).

2-3. Identification of Signaling Factors Controlling Stimulatory Effect of Vascular Endothelial Cells

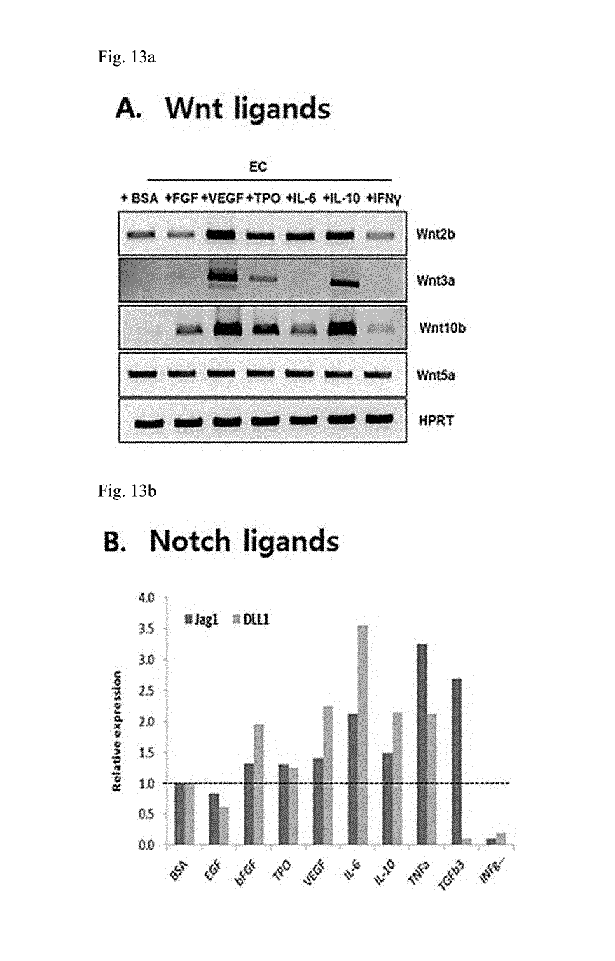

In the above-described examples, the function and related mediators of vascular endothelial cells stimulating mesenchymal stem cells were identified. In this example, as a means for controlling the ability of vascular endothelial cells to stimulate mesenchymal stem cells, an upstream signaling system which controls related activity of the vascular endothelial cells was analyzed. As a result, the expression of a Wnt ligand in vascular endothelial cells, indicating that vascular endothelial cells serve as mediators for stimulating mesenchymal stem cells, was increased due to bFGF, VEGF, TPO, IL-6, and IL-10, and bFGF, VEGF, IL-6, IL-10 and TNF-alpha were involved in a significant increase in the expression of notch ligands jagged-1 and D11-1 in mesenchymal stem cells (FIGS. 13a and 13b).

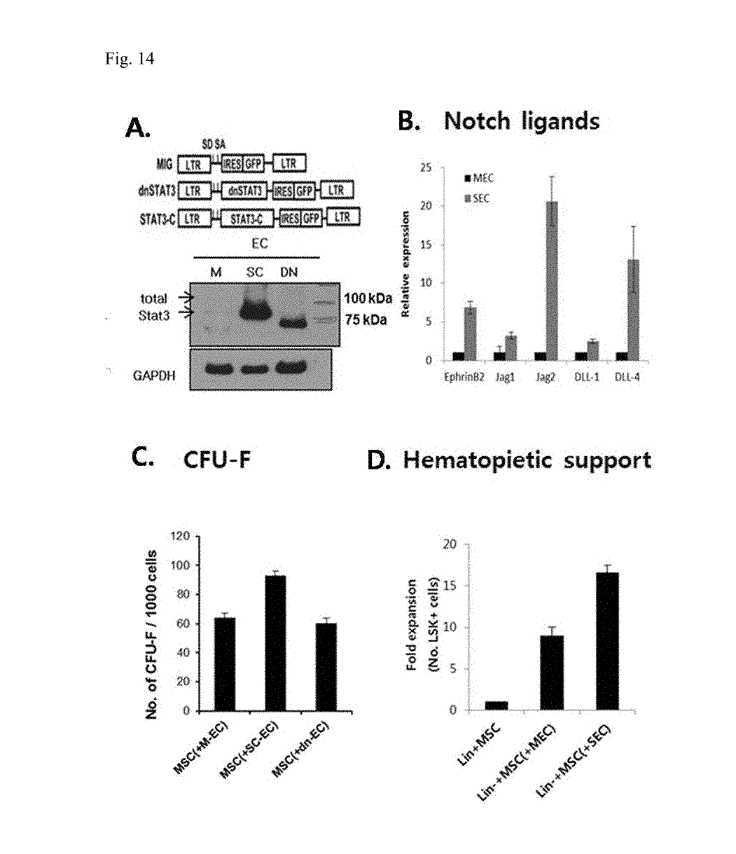

In addition, it can be seen that STAT3 activation served as a major factor for activating vascular endothelial cells by the cytokines. Specifically, retrovirus vectors expressing activated STAT3 (STAT3-C; SC, the sequence disclosed in Bromberg J F, Wrzeszczynska M H, Devgan G, et al. Stat3 as an oncogene. Cell. 1999; 98:295-303) and inhibited STAT3 (dnSTAT3) were constructed (FIG. 14A), and then introduced into vascular endothelial cells to express the activated STAT3 and inhibited STAT3, confirming that in the activated STAT3-introduced vascular endothelial cells, notch ligands such as Jgged-1, 2 and Dll-4 as well as an EphrinB2 ligand were clearly increased (FIG. 14B). Moreover, as a result of co-culture of each type of the vascular endothelial cells expressing activated STAT3 or inhibited STAT3 and mesenchymal stem cells, it was confirmed that CFU-Fs of mesenchymal stem cells were increased by STAT3-C (FIG. 14C). Particularly, mesenchymal stem cells in contact with each type of vascular endothelial cells were isolated by sorting, and then co-cultured with undifferentiated hematopoietic stem cells (Lin−), thereby inducing the expansion of hematopoietic stem cells (Lin−Sca-1+c-kit+; LSK) in the mesenchymal stem cells (Lin+MSC(+MEC)) in contact with the vascular endothelial cells, which was much higher than that of single-cultured mesenchymal stem cells (Lin+MSC), and particularly, mesenchymal stem cells (Lin+MSC(+SEC)) in contact with the vascular endothelial cells (SEC) in which STAT3-C was activated showed a higher hematopoietic stem cell expansion capacity than that of the mesenchymal stem cells in contact with the other type of vascular endothelial cells (FIG. 14D).

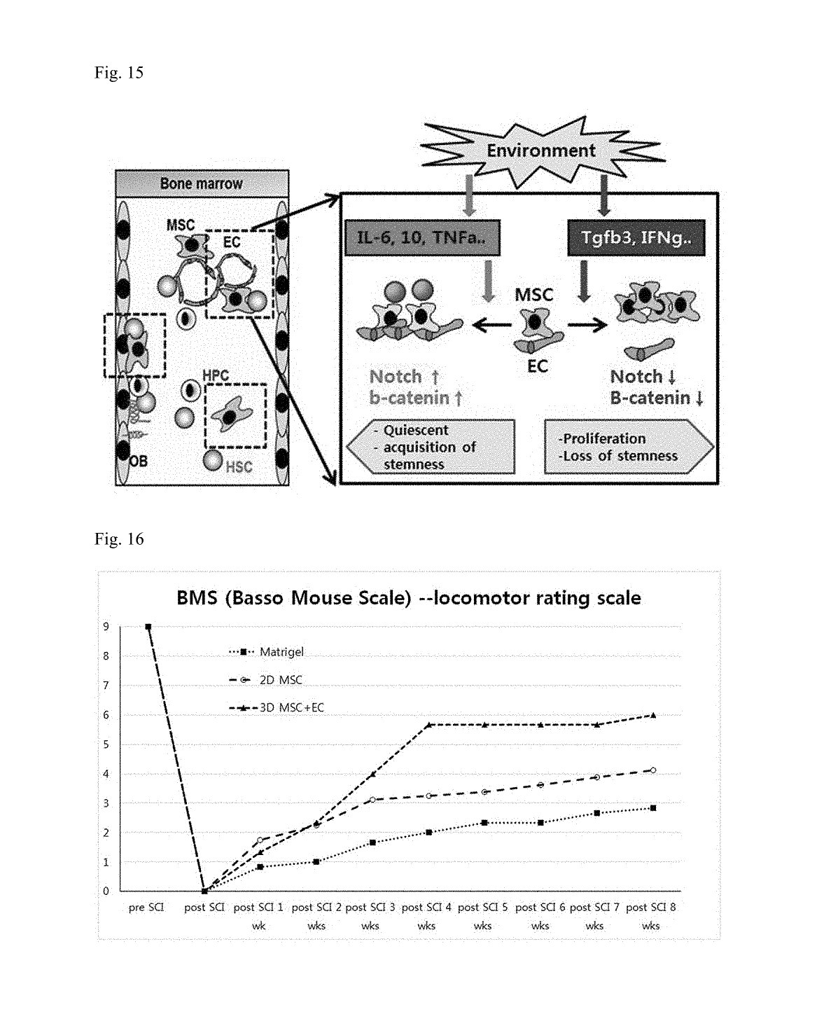

In other words, it was confirmed that cytokines such as IL-6 and IL-10 serve to activate vascular endothelial cells (STAT3, the Wnt ligand and the EphrinB2 ligand), and the mesenchymal stem cells in contact with the activated vascular endothelial cells were enhanced in niche activity (b-catenin, notch), resulting in the induction of self-renewal and expansion of undifferentiated hematopoietic stem cells (FIG. 15). Therefore, the present invention identified that the niche activity of the mesenchymal stem cells and the self-renewal capacity of the hematopoietic stem cells can be up- or down-regulated by controlling the expression of the activity of the vascular endothelial cells and the expression of related factors.

### Example 3. Confirmation of Effect of Promoting the Self-Renewal of Neural Stem Cells

In this embodiment, it was confirmed whether an interaction between vascular endothelial cells and mesenchymal stem cells can affect the self-renewal of the nervous system as well as the hematopoietic system.

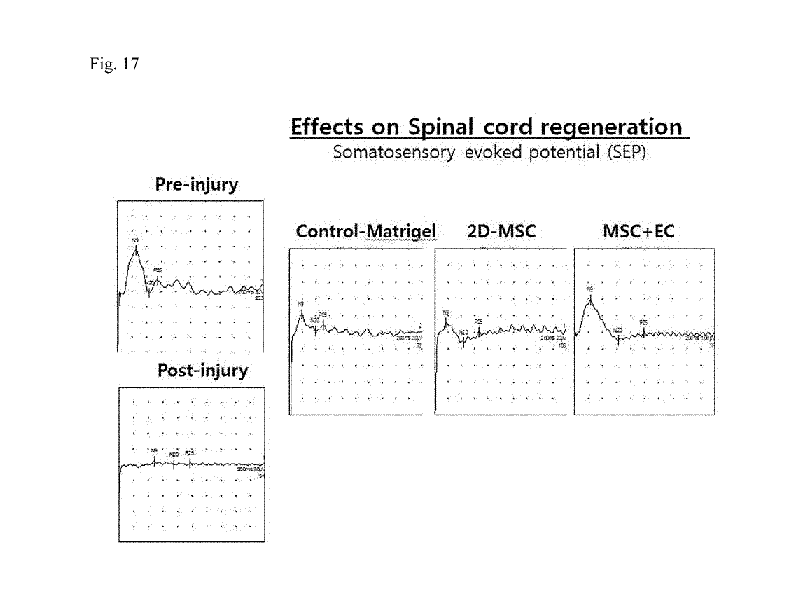

After each type of cells (2D-MSC or 3D MSC+EC) were mixed with Matrigel and administered to a damage site of a spinal cord-cut mouse model, the recovery of the spinal cord function was compared and analyzed using a locomotor rating scale. By the assessment using a Basso Mouse Scale (BMS) method for measuring the function of the motor nerves, compared to the Matrigel only-administered group, the mesenchymal stem cell-administered group (2D-MSC) exhibited a higher locomotor recovery, and particularly, it was confirmed that the mesenchymal stem cell+vascular endothelial cell-administered group (3D MSC+EC) exhibited a considerably higher level of locomotor recovery (FIG. 16). In the analysis using a somatosensory evoked potential (SEP) method for measuring the function of sensory nerves, it was observed that the functionality in the MSC+EC group was also increased similar to that described above (FIG. 17).

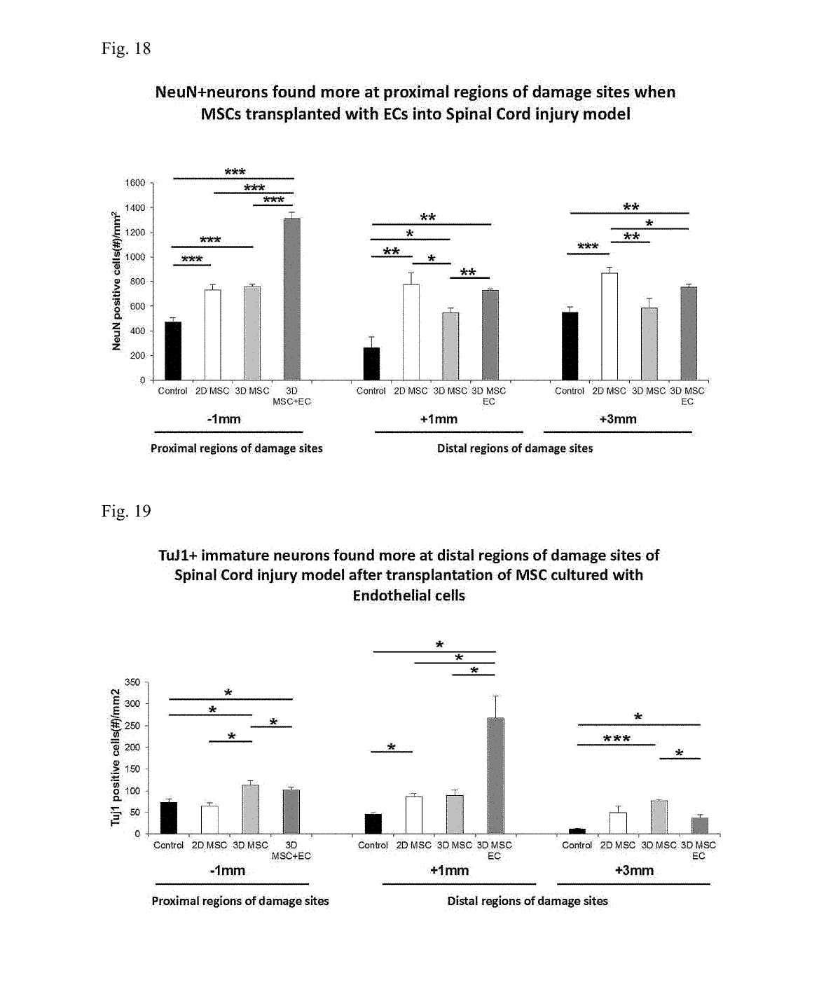

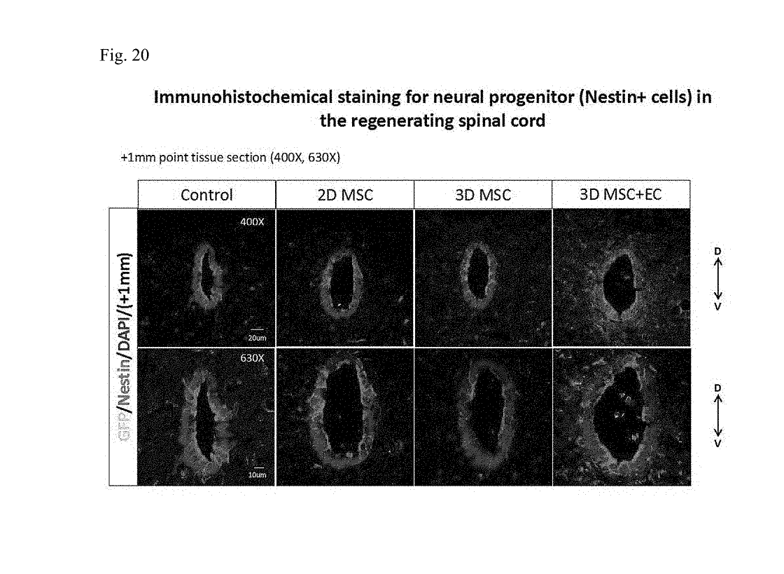

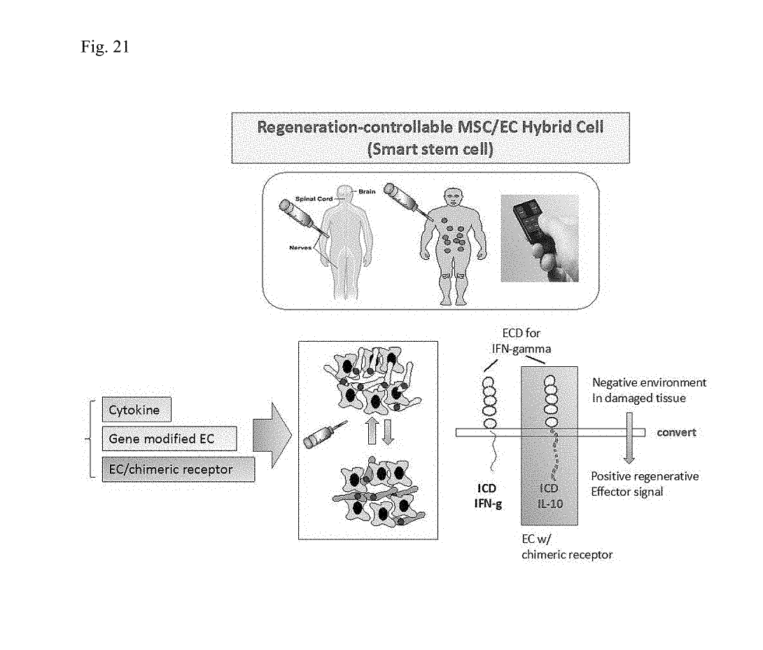

In addition, spinal cord tissue that is in a regenerated state was analyzed by immunofluorescent staining, and as control groups, the mesenchymal stem cell only-administered group (2D-MSC) and the three-dimensionally-cultured mesenchymal stem cell-administered group (3D-MSC) was used. It was confirmed that, in the group (3D MSC+EC) in which the mesenchymal stem cells were mixed with the vascular endothelial cells and administered, NeuN-positive cells, which are a marker for nerve cells in a distal (−1 mm) region of the spinal cord-cut damage site, and Tuj-1-positive cells, which is a marker for nerve cells in a proximal (+1 mm) region of the spinal cord-cut damage site, were significantly increased, compared to other comparative groups (FIGS. 18 and 19). In addition, it was confirmed that nestin-positive cells, which are a marker corresponding to regeneration-associated neural stem cells (neural progenitor) as well as differentiated nerve cell tissue, were significantly increased, compared to other comparative groups (FIG. 20). These results show that the interaction between the vascular endothelial cells and the mesenchymal stem cells also greatly affects the self-renewal of the nervous system.

It should be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments described above are exemplary in all aspects, and are not limitative.

What is claimed is:

1. A composition for promoting the self-renewal of adult stem cells comprising vascular endothelial cells over-expressing any one or more of ligands Wnt and EphrinB2.

2. The composition according to claim 1, wherein the vascular endothelial cells are increased in STAT3 activity.

3. The composition according to claim 1, wherein vascular endothelial cells are transfected with a vector including any one of a Wnt ligand, an EphrinB2 ligand, activated STAT3 (STAT3-C) and a combination thereof.

4. The composition according to claim 1, wherein vascular endothelial cells are treated with any one factor selected from a basic fibroblast growth factor (b-FGF), thrombopoietin (TPO), a vascular endothelial growth factor (VEGF), interleukin-6 (IL-6), interleukin-10 (IL-10), tumor necrosis factor-α (TNF-α) and a combination thereof.

5. The composition according to claim 1, wherein the adult stem cells are hematopoietic stem cells or neural stem cells.

6. The composition according to claim 1, further comprising ex-vivo isolated mesenchymal stem cells, which are provided in physical contact with the vascular endothelial cells.

7. A composition for promoting the self-renewal of adult stem cells comprising vascular endothelial cells; and any one factor selected from a basic fibroblast growth factor (b-FGF), thrombopoietin (TPO), a vascular endothelial growth factor (VEGF), interleukin-6 (IL-6), interleukin-10 (IL-10), tumor necrosis factor-α (TNF-α) and a combination thereof.

8. The composition according to claim 7, wherein the adult stem cells are hematopoietic stem cells or neural stem cells.

9. The composition according to claim 7, further comprising ex-vivo isolated mesenchymal stem cells, which are provided in physical contact with the vascular endothelial cells.

10. A method of preparing mesenchymal stem cells with enhanced niche activity comprising co-culturing vascular endothelial cells and mesenchymal stem cells, which are ex-vivo isolated, in physical contact with each other.

11. The method according to claim 10, wherein, before the co-culturing, further comprising over-expressing any one of a Wnt ligand, an EphrinB2 ligand, activated STAT3 (STAT3-C) and a combination thereof in vascular endothelial cells.

12. The method according to claim 10, wherein the co-culturing comprises adding any one factor selected from the group consisting of a basic fibroblast growth factor (b-FGF), thrombopoietin (TPO), a vascular endothelial growth factor (VEGF), interleukin-6 (IL-6), interleukin-10 (IL-10), tumor necrosis factor-α (TNF-α) and a combination thereof.

13. The method according to claim 10, wherein the mesenchymal stem cells are derived from adipose tissue, bone marrow, peripheral blood or umbilical cord blood.

14. A method of regulating the niche activity of mesenchymal stem cells comprising over- or under-expressing any one or more of ligands Wnt and EphrinB2 in vascular endothelial cells, which are co-cultured with mesenchymal stem cells while being in physical contact therewith.

15. The method according to claim 14, wherein the over-expressing comprises transfecting vascular endothelial cells with a vector including any one of a Wnt ligand, an EphrinB2 ligand, activated STAT3 (STAT3-C) and a combination thereof.

16. The method according to claim 14, wherein the over-expressing comprises treating vascular endothelial cells with a factor selected from the group consisting of a basic fibroblast growth factor (b-FGF), thrombopoietin (TPO), a vascular endothelial growth factor (VEGF), interleukin-6 (IL-6), interleukin-10 (IL-10), tumor necrosis factor-α (TNF-α) and a combination thereof.

17. The method according to claim 14, wherein the under-expressing comprises treating vascular endothelial cells with a transforming growth factor, beta 3 (Tgfβ3), interferon gamma (IFN-γ) and a combination thereof.
